(19) United States
(12) Patent Application Publication
(10) Pub. No.: US 2016/0331675 A1
(43) Pub. Date: Nov. 17, 2016

(54) HAIR CONDITIONING COMPOSITION

(71) Applicant: Theresa Jamerson, McKinney, TX (US)

(72) Inventor: Theresa Jamerson, McKinney, TX (US)

(21) Appl. No.: 14/710,671

(22) Filed: May 13, 2015

(51) Int. Cl. A61K 8/97; A61Q 5/12

(52) U.S. Cl. CPC A61K 8/97; A61Q 5/12

(57) ABSTRACT

A hair conditioning composition for moisturizing hair includes a mixture containing Shea butter, Kokum butter, coconut oil, avocado oil, olive oil, grape seed oil, almond oil, wheat germ oil, flaxseed oil, jojoba oil, castor oil, aloe vera gel and orange oil.

### BACKGROUND OF THE DISCLOSURE

1. Field of the Disclosure

The disclosure relates to hair conditioning mixtures and more particularly pertains to a new hair conditioning mixture for promoting healthy, moisturized hair.

### SUMMARY OF THE DISCLOSURE

An embodiment of the disclosure meets the needs presented above by generally comprising a mixture containing Shea butter, Kokum butter, coconut oil, avocado oil, olive oil, grape seed oil, almond oil, wheat germ oil, flaxseed oil, jojoba oil, castor oil, aloe vera gel and orange oil.

There has thus been outlined, rather broadly, the more important features of the disclosure in order that the detailed description thereof that follows may be better understood, and in order that the present contribution to the art may be better appreciated.

There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### DESCRIPTION OF THE PREFERRED EMBODIMENT

A new hair conditioning mixture embodying the principles and concepts of an embodiment of the disclosure will be described.

The hair conditioning composition generally comprises a mixture containing Shea butter, Kokum butter, coconut oil, avocado oil, olive oil, grape seed oil, almond oil, wheat germ oil, flaxseed oil, jojoba oil, castor oil, aloe Vera gel and orange oil. The mixture is substantially free of water and this case that is defined as having no or very little water added to the mixture such that the water weight of the composition is less than 5%, preferably less than 3% and more preferably less than 1%. It is understood that the components of the mixture may themselves contain some water but the composition will be blended using these components in their natural, readily obtainable condition and no water will typically be added to the composition.

The composition may more particularly comprise:
35.0-45.0% Shea butter (fat from the nut of the Shea tree, Vitellaria paradoxa);
17.0-23.0% Kokum butter (oil from the kokum tree, Garcinia indica);
8.0-12.0% coconut oil (oil extracted from coconuts from the coconut palm, Cocos nucifera);
4.0-6.0% avocado oil (oil pressed from the fruit of an avocado plant, Persea americana);
1.5-3.5% olive oil (oil from olive trees, Olea europaea);
1.5-3.5% grape seed oil (oil from pressed seeds of grapes, various grapes from the Vitis family);
1.5-3.5% almond oil (oil from the nuts of almond trees, such as Prunus dulcis);
1.5-3.5% wheat germ oil (extracted oil from the germ of a wheat kernel, any of a variety from the Genus Triticum);
1.5-3.5% flaxseed oil (oil obtained from seeds of the flax plant, Linum usitatissimum);
1.5-3.5% jojoba oil (oil from the seed of the jojoba plant, Simmondsia chinensis);
1.5-3.5% castor oil (oil form the seeds of the castor oil plant, Ricinus communis);
1.5-3.5% aloe vera gel (extracts from the Aloe vera plant); and
5.0-9.0% orange oil (oil extracted from the rind of the fruit of an orange tree, Citrus sinensis).

A more specific embodiment of the composition may further comprise:
39.16% Shea butter;
19.58% Kokum butter;
9.79% coconut oil;
4.90% avocado oil:
2.45% olive oil;
2.45% grape seed oil;
2.45% almond oil;
2.45% wheat germ oil;
2.45% flaxseed oil;
2.45% jojoba oil;
2.45% castor oil;
2.45% aloe vera gel; and
6.99% orange oil.

The components above are simply mixed together and such may take place in high speed blending machine to thoroughly incorporate the components into each other. Once the composition is made, it may be stored in conventional containers of plastic, glass and the like. To use, the composition is simply added to the person's hair and then rinsed out.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A hair conditioning composition comprising:
a mixture containing Shea butter, Kokum butter, coconut oil, avocado oil, olive oil, grape seed oil, almond oil, wheat germ oil, flaxseed oil, jojoba oil, castor oil, aloe vera gel and orange oil.

2. The hair conditioning composition according to claim 1, wherein said mixture is substantially free of water.

3. The hair conditioning composition according to claim 2, wherein said mixture contains by weight:
35.0-45.0% Shea butter;
17.0-23.0% Kokum butter;
8.0-12.0% coconut oil; and
4.0-6.0% avocado oil.

4. The hair conditioning composition according to claim 2, wherein said mixture contains by weight 5.0-9.0% orange oil.

5. A hair conditioning composition consisting essentially of Shea butter, Kokum butter, coconut oil, avocado oil, olive oil, grape seed oil, almond oil, wheat germ oil, flaxseed oil, jojoba oil, castor oil, aloe vera gel and orange oil.

6. A hair conditioning composition comprising:
a mixture containing by weight:
35.0-45.0% Shea butter;
17.0-23.0% Kokum butter;
8.0-12.0% coconut oil;
4.0-6.0% avocado oil;
1.5-3.5% olive oil;
1.5-3.5% grape seed oil;
1.5-3.5% almond oil;
1.5-3.5% wheat germ oil;
1.5-3.5% flaxseed oil;
1.5-3.5% jojoba oil;
1.5-3.5% castor oil;
1.5-3.5% aloe vera gel;
5.0-9.0% orange oil; and
said mixture being substantially free of water.
